SEQUENCE LISTING (4 sequences)

SEQ ID NO: 1 - protein, 6 aa, synthetic construct
YILDCN

SEQ ID NO: 2 - protein, 11 aa, synthetic construct
YGRKKRRQRRR

SEQ ID NO: 3 - protein, 17 aa, synthetic construct
YGRKKRRQRRRYILDCN

SEQ ID NO: 4 - protein, 17 aa, synthetic construct
YGRKKRRQRRRCNDILY

(19) United States
(12) Patent Application Publication
(10) Pub. No.: US 2026/0042799 A1
(43) Pub. Date: Feb. 12, 2026

(54) APPLICATION OF PEPTIDE IN PREPARING DRUGS FOR TREATING AND/OR PREVENTING COGNITIVE IMPAIRMENT

(71) Applicant: HUAZHONG UNIVERSITY OF SCIENCE AND TECHNOLOGY, Hubei (CN)

(72) Inventors: Lingqiang ZHU, Hubei (CN); Dan LIU, Hubei (CN); Xiya SHEN, Hubei (CN); Hezhou HUANG, Hubei (CN); Liunan YANG, Hubei (CN)

(73) Assignee: HUAZHONG UNIVERSITY OF SCIENCE AND TECHNOLOGY, Hubei (CN)

(21) Appl. No.: 19/366,471

(22) Filed: Oct. 22, 2025

Related U.S. Application Data
(63) Continuation of application No. PCT/CN2024/092157, filed on May 10, 2024.

(30) Foreign Application Priority Data
Apr. 26, 2024 (CN) 202410513821.9

(51) Int. Cl. C07K 7/06; A61K 38/00; A61P 25/28

(52) U.S. Cl. CPC C07K 7/06; A61P 25/28; A61K 38/00; C07K 2319/10

(57) ABSTRACT

The present disclosure relates to an application of a peptide in preparing drugs for treating and/or preventing cognitive impairment, belonging to the technical field of drugs for cognitive impairment. The present disclosure discloses an application of an artificially synthesized small molecule peptide in preparing drugs for treating and/or preventing cognitive impairment. When the peptide in the present disclosure is applied to cognitive impairment models, it may effectively play its biological role in blocking the binding between Synapsin IIb (Syn2b) and AMPA receptor subunit (GluA2), enabling the increase of GluA2 membrane expression, increase of AMPA current, enhancement of neuronal excitability and synaptic transmission, thereby alleviating learning and memory impairment in cognitive impairment model mice to some extent.

### CROSS-REFERENCE TO RELATED APPLICATION

This application is a continuation of international application of PCT application serial no. PCT/CN2024/092157, filed on May 10, 2024, which claims the priority benefit of China application no. 202410513821.9 filed on Apr. 26, 2024. The entirety of each of the above-mentioned patent applications is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Mar. 11, 2025, is named 154461-US-Sequence_List and is 4,489 bytes in size.

### Technical Field

The present disclosure relates to drugs for treating cognitive impairment, more specifically, relates to an application of a peptide in preparing drugs for treating and/or preventing cognitive impairment.

### Description of Related Art

Alzheimer's disease (AD) is a progressively developing neurodegenerative disorder of the central nervous system with an insidious onset, predominantly affecting the elderly population. AD represents the most common form of dementia and has emerged as the highest incidence disease among all dementia typologies, constituting the second most prevalent life-threatening condition in the elderly after cardiovascular diseases. The principal symptomatic manifestation of AD is characterized by progressive memory deterioration, with short-term memory impairment typically presenting as the primary initial symptom, ultimately progressing to long-term memory dysfunction until death. This disorder severely impairs the patient's capacity for normal functioning, causing substantial suffering to the afflicted individuals and imposing significant burdens on families and society. Furthermore, AD exerts considerable emotional and psychological impact on both patients and their immediate relatives. Therefore, in light of the increasingly prominent issue of population aging, research into the pathological foundations of AD and the development of preventative pharmaceutical interventions have become matters of urgent necessity.

Neural synapses constitute the functional junctions between neurons, serving as critical sites for information transmission and neuronal communication. Synapses represent the fundamental units of the brain, wherein synaptic activity stimulates the maturation of mushroom-shaped dendritic spines and facilitates the formation of new synaptic connections. This enables synaptic strength to adapt to environmental changes, thereby playing a vital role in learning and memory processes. Synapsins include a family of neuron-specific phosphoproteins associated with synaptic vesicles that play significant roles in regulating neurotransmitter release and early neuronal development. Synapsin II (Syn2), a member of the Synapsin (I/II/III) protein family, undergoes alternative splicing to produce two protein isoforms: Syn2a (547 amino acids) and Syn2b (478 amino acids). These isoforms participate in vesicle anchoring, release, and neurotransmission processes. Research utilizing co-immunoprecipitation techniques has demonstrated that Syn2b, but not Syn2a, binds to glutamate receptor 2 (GluA2), thereby influencing AMPA receptor membrane trafficking. In the brain, AMPA-type glutamate receptors (AMPARs) form complexes with auxiliary subunits to mediate the majority of rapid excitatory neurotransmission. Studies involving adeno-associated virus-mediated overexpression of Syn2b protein in the medial prefrontal cortex (mPFC) have recorded excitatory postsynaptic currents, revealing significantly decreased amplitudes in neurons overexpressing Syn2b. This suggests that Syn2b may influence learning and memory-related mechanisms through regulation of AMPA receptor surface expression. Mounting evidence indicates that excitatory amino acids, particularly glutamate, play crucial roles in the pathogenesis of AD. AMPA receptors and their subunits are involved in various aspects of synaptogenesis, synaptic extension, and synaptic plasticity—the physiological basis for learning and memory processes. Consequently, AMPA receptor dysfunction is particularly implicated in the pathogenesis of numerous neurological disorders, including AD, frontotemporal dementia, and Parkinson's disease.

TAT, known as cell penetrating peptides, is a highly efficient transport vector discovered in recent years. TAT can penetrate cell membranes and nuclear membranes, carrying peptides, proteins, and DNA molecules through receptor-mediated transport into the cytoplasm and nucleus to exert corresponding biological effects. Current research indicates that HIV-TAT can penetrate all tissue cells without significant toxic side effects. TAT can transport connected peptides into cells within minutes and can cross the blood-brain barrier to enter neurons. The peptides transported into cells retain their original biological activity, thereby exerting their biological functions.

### SUMMARY

The purpose of the present disclosure lies in providing a small molecule peptide P6, with its sequence shown in sequence table SEQ ID NO.1. Further, a membrane peptide with the sequence shown in SEQ ID NO.2 is connected to the N-terminus of P6. The present disclosure further connects the TAT membrane peptide (YGRKKRRQRRR, i.e., SEQ ID NO.2) with P6 (YILDCN, i.e., SEQ ID NO.1) to obtain the biologically active TAT-P6 peptide (YGRKKRRQRRRYILDCN, i.e., SEQ ID NO.3), utilizing membrane penetration function of TAT to deliver the P6 peptide into the blood and through the blood-brain barrier, to be taken up by brain neurons, thereby playing its biological function.

According to the first aspect of the present disclosure, an application of a peptide in preparing drugs for treating and/or preventing cognitive impairment is provided, wherein the amino acid sequence of the peptide is shown in SEQ ID NO:1.

Preferably, the cognitive impairment is perception impairment, memory impairment, or thinking impairment.

Preferably, the perception impairment is sensory hypersensitivity, sensory dullness, internal sensory discomfort, sensory degeneration, sensory deprivation, pathological illusion or hallucination, or comprehensive perception impairment.

The memory impairment is memory hypermnesia, memory deficit, or memory error.

The thinking impairment is abstract generalization process impairment, association process impairment, logical thinking impairment, or delusion.

Preferably, the cognitive impairment is cognitive impairment caused by Alzheimer's disease, Lewy body dementia, frontotemporal dementia, Parkinson's disease, or Huntington's chorea.

Or, the cognitive impairment is cognitive impairment after traumatic brain injury, cognitive impairment after cerebrovascular disease cognitive damage, or postoperative cognitive impairment.

Or, the cognitive impairment is cognitive impairment caused by progressive supranuclear palsy, corticobasal degeneration, argyrophilic grain disease, Pick's disease, neurasthenia, hysteria, skepticism, menopausal syndrome, depression, obsessive-compulsive disorder, schizophrenia, reactive psychosis, paranoid psychosis, mania, or manic depression.

Preferably, the peptide is used to block the binding of Syn2b protein with GluA2 protein, thereby enabling the increase of GluA2 protein membrane expression.

Preferably, the peptide is used to promote the increase of neuronal AMPA current, thereby enabling the increase of spontaneous excitatory postsynaptic current.

Preferably, the N-terminus of the peptide is further connected with a membrane peptide.

Preferably, the amino acid sequence of the membrane peptide is as shown in SEQ ID NO: 2.

According to another aspect of the present disclosure, a peptide prepared for treating and/or preventing cognitive impairment is provided, wherein the amino acid sequence of the peptide is as shown in SEQ ID NO:1.

Preferably, the N-terminus of the peptide is further connected with a membrane peptide.

Preferably, the amino acid sequence of the membrane peptide is as shown in SEQ ID NO: 2.

Overall, compared with the related art, the technical solutions conceived by the present disclosure mainly have the following technical advantages.
(1) The present disclosure provides a membrane-permeable small molecule peptide P6 composed of only 6 amino acids, with its sequence as shown in sequence table SEQ ID NO.1, which improves learning memory ability in cognitive impairment mice (such as AD mice) by reducing the binding between Syn2b and GluA2. Furthermore, a membrane peptide with the sequence shown in SEQ ID NO.2 is connected to the N-terminus of P6. The present disclosure further connects the TAT membrane peptide (YGRKKRRQRRR, i.e., SEQ ID NO.2) with P6 (YILDCN, i.e., SEQ ID NO.1) to obtain the biologically active TAT-P6 peptide (YGRKKRRQRRRYILDCN, i.e., SEQ ID NO.3), utilizing the membrane-penetrating function of TAT to deliver the P6 peptide into the blood and through the blood-brain barrier, and be taken up by brain neurons to play its biological function.
(2) The small molecule peptide TAT-P6 involved in the present disclosure has high purity, is completely soluble, suitable for subcutaneous injection, and has no toxic side effects.
(3) In the TAT-P6 peptide disclosed in the present disclosure, TAT may carry the P6 protein peptide through the blood-brain barrier via blood to be taken up by neurons, which may be transformed and applied to the nervous system, regulating learning memory in Alzheimer's disease, making it practically feasible.
(4) The present disclosure preferably applies the peptide in the present disclosure to the therapeutic effect on learning memory impairment in AD model mice, through subcutaneous injection, effectively blocking the binding between Syn2b and GluA2, restoring the learning memory ability of AD model mice, providing a method for improving the adverse effects caused by AD.

### BRIEF DESCRIPTION OF THE DRAWINGS

FIG. 1 is a mass spectrometry analysis diagram of an artificially synthesized small molecule peptide TAT-P6.

FIG. 2 shows the immunoprecipitation diagram of TAT-P6 peptide and control peptide TAT-S6 disrupting the binding between HA-GluA2 and EGFP-Syn2b in HEK293T cells co-transfected with HA-GluA2 and EGFP-Syn2b overexpression plasmids. First, HA-GluA2 and EGFP-Syn2b overexpression plasmids were transfected into HEK293T cells. After 48 hours of transfection, 10 μM concentration of TAT-P6 and TAT-S6 peptides were given in the cell culture medium for 90 minutes, TAT-P9 adds three amino acids (CLQYILDCN) to the N-terminus of P6, and then cells were collected. The overexpressed Syn2b protein is a fusion protein of Syn2b and GFP, using anti-GFP antibody to precipitate cellular proteins, and then using anti-GluA2 antibody to detect the precipitated proteins, the black imprint on the NC membrane indicates the interaction between GluA2 and Syn2b. After giving 10 μM TAT-P6 and TAT-P9, the anti-GluA2 antibody could hardly detect the corresponding protein on the NC membrane, suggesting that TAT-P6 blocked the interaction between Syn2b and GluA2. In the control group given TAT-S6, GluA2 protein could be clearly detected, indicating that TAT-S6 could not block the interaction between Syn2b and GluA2.

FIG. 3A to FIG. 3J show the statistical chart of TAT-P6 peptide improving mouse learning memory ability results. FIGS. 3A-B are behavioral experiments for four groups of mice (C57 mice injected with TAT-S6 group, C57 mice injected with TAT-P6 group, P301S mice injected with TAT-S6 group, P301S mice injected with TAT-P6 group) using open field test system. FIG. 3A shows the statistical result chart of the total movement distance of four groups of mice in the open field experiment. There was no statistical significance in the total movement distance between C57 mice injected with TAT-S6 peptide solution and C57 mice given TAT-P6 peptide solution injection. There was no statistical significance in the total movement distance between P301S mice injected with TAT-S6 peptide solution and P301S mice given TAT-P6 peptide solution injection. FIG. 3B shows the statistical result chart of the time spent in the center position by four groups of mice in the open field experiment. There was no statistical significance in the time spent in the center position between C57 mice injected with TAT-S6 peptide solution and C57 mice given TAT-P6 peptide solution injection. There was no statistical significance in the time spent in the center position between P301S mice injected with TAT-S6 peptide solution and P301S mice given TAT-P6 peptide solution injection. This indicates that TAT-S6 did not change the movement ability of mice. FIG. 3C shows behavioral experiments for four groups of mice using novel object recognition test system, where C data indicates that P301S mice injected with TAT-S6 peptide solution spent less time at the new object, while in comparison, P301S mice given TAT-P6 peptide solution injection spent more time at the new object. FIGS. 3D-H are behavioral experiments for four groups of mice using Morris water maze test system, where FIG. 3D data shows the movement trajectory of four groups of mice on the 8th day of the water maze experiment, and FIG. 3E shows the statistical result chart of the latency period during the training stage of the first 6 days of the experiment. FIG. 3F shows the statistical result chart of the latency period of four groups of mice in the water maze at the detection stage on the 8th day. P301S mice injected with TAT-S6 peptide solution had prolonged latency to reach the platform, while P301S mice given TAT-P6 peptide solution injection had significantly reduced latency to reach the platform. FIG. 3G shows the statistical result chart of the number of times four groups of mice crossed the quadrant where the platform was located in the water maze at the detection stage on the 8th day. P301S mice injected with TAT-S6 peptide solution crossed the platform significantly fewer times than P301S mice given TAT-P6 peptide solution injection. This indicates that injection of TAT-P6 peptide can effectively improve the learning memory function of P301S mice. FIG. 3H shows the statistical result chart of the time spent in the target quadrant by four groups of mice in the water maze at the detection stage on the 8th day. P301S mice injected with TAT-S6 peptide solution spent significantly less time in the target quadrant than P301S mice given TAT-P6 peptide solution injection. This indicates that injection of TAT-P6 peptide can effectively improve the learning memory function of P301S mice. FIG. 3I shows the percentage (%) of freezing time of four groups of mice in the contextual fear conditioning experiment. FIG. 3J shows the percentage (%) of freezing time of four groups of mice in the cued fear conditioning experiment after changing the environment with sound stimulation.

FIG. 4A and FIG. 4B shows the statistical chart of immunoblotting results indicating that TAT-P6 peptide does not change the total protein amount of Syn2b and GluA2. Using the proteins prepared above, the content of Syn2b and GluA2 in TAT-P6 peptide group and control group mice were detected respectively, with GAPDH as the loading control proteins, proving the consistency of protein loading amount. Through comparison, it was discovered that mice given subcutaneous injection of 15 mg/kg TAT-P6 solution showed no significant change in protein content of Syn2b and GluA2 compared with control group mice.

FIG. 5 shows the statistical chart of immunoblotting results indicating that TAT-P6 peptide enables the increase of GluA2 membrane expression. After the behavioral tests were completed, membrane proteins from the hippocampal brain region of mice were isolated. Through immunoblotting, it was discovered that the GluA2 protein on the membrane of mice in the TAT-P6 peptide solution treatment group was significantly higher than that of the control group, with ATP1A1 as the loading control proteins for membrane protein.

FIG. 6A to FIG. 6C shows the statistical chart of results indicating that TAT-P6 peptide enhances AMPA current in mouse neurons. C57 mice and P301S mice were euthanized by cervical dislocation after anesthesia, brain tissues were rapidly removed and placed in frozen artificial cerebrospinal fluid (aCSF). According to experimental requirements, the hippocampal brain region was isolated, and the tissue was cut into 300 um thick slices using a vibratome, followed by incubation at room temperature for 1 h. Subsequently, 10 μM TAT-P6 solution or 10μM TAT-S6 solution was given in the perfusion solution for 30 min treatment. Appropriate stimulation and recording electrode positions were selected to record AMPA current and NMDA current in the hippocampal brain region, with 10 cells recordable for each brain slice. The experiment discovered that after treatment with 10 μM TAT-P6 solution, compared with incubation with 10 μM TAT-S6 solution, the AMPA current amplitude increased, while NMDA current showed no significant change, and the NMDA/AMPA ratio significantly decreased.

FIG. 7A and FIG. 7B shows the statistical chart of results indicating that TAT-P6 peptide enhances mouse neuron excitability. Spontaneous excitatory postsynaptic currents (sEPSC) in the hippocampal brain region of P301S mice were recorded, with 10 cells recordable for each brain slice. The experiment discovered that after treatment with 10 μM TAT-P6 solution, compared with incubation with 10 μM TAT-S6 solution, the amplitude of sEPSC increased, proving enhanced neuron excitability.

### DESCRIPTION OF THE EMBODIMENTS

In order to make the purpose, technical solution and advantages of the present disclosure more comprehensible, the following further detailed explanation of the present disclosure is provided in combination with the drawings and embodiments. It should be understood that the specific embodiments described here are merely used to explain the present disclosure, and not to limit the present disclosure. In addition, the technical features involved in various embodiments of the present disclosure described below may be combined with each other as long as they do not conflict with each other.

The purpose of the present disclosure lies in providing a small molecule peptide P6, with

its sequence shown in sequence table SEQ ID NO.1. Further, a membrane peptide with the sequence shown in SEQ ID NO.2 is connected to the N-terminus of P6. The present disclosure further connects the TAT membrane peptide (YGRKKRRQRRR, i.e., SEQ ID NO.2) with P6 (YILDCN, i.e., SEQ ID NO.1) to obtain the biologically active TAT-P6 peptide (YGRKKRRQRRRYILDCN, i.e., SEQ ID NO.3), utilizing membrane penetration function of TAT to deliver the P6 peptide into the blood and through the blood-brain barrier, to be taken up by brain neurons, thereby playing its biological function. The control for TAT-P6 is TAT-S6, with its sequence shown in SEQ ID NO.4, as follows: YGRKKRRQRRRCNDILY.

The present disclosure preferably provides a small molecule peptide TAT-P6, and the application of this peptide in improving cognitive impairment, further in the application for Alzheimer's disease (AD), and more further in the application for improving learning and memory behavior in Alzheimer's disease. When administered through subcutaneous injection, it was discovered that it may effectively play its biological role in blocking the binding between Syn2b and GluA2, enabling increased membrane expression of GluA2, increased AMPA current, elevated neuron excitability, and enhanced synaptic transmission, thereby restoring the learning and memory ability of AD mouse models to some extent.

In the present disclosure, the TAT-P6 peptide and control TAT-S6 were synthesized and prepared by the applicant through commissioning Qiangyao Biotechnology Co., Ltd.

### Example 1

### Materials for Experiment

The present disclosure, through randomly selecting P301S mice and C57/BL6 for

subcutaneous injection of TAT-P6 solution, may block the binding between GluA2 and Syn2b, and may improve learning and memory impairment in AD mice.

The specific operation steps are as follows:

### I. Application of TAT-P6 Peptide in Improving Cognitive Impairment

### 1. Experimental Subjects

Male P301S mice 6-8 months old (purchased from Jackson Laboratory), C57/BL6 mice 6-8 months old (purchased from Beijing Vital River Laboratory Animal Technology Co., Ltd.), SPF grade, weight 20-25 grams, raised in conventional environment.

### 2. Research Methods

1) Mouse spatial memory detection: novel object recognition and water maze;
a) Novel object recognition: Before conducting the novel object recognition test, it is necessary to eliminate unfamiliarity with the mice by petting them daily to prevent stimulation during operation. The first stage is adaptation period. Mice move freely in the experimental device (without objects) for 10 minutes. The second stage is familiarization period. Two identical objects are placed in the device (AB, ensuring objects have no odor and cannot be pushed), with objects 10 cm away from both side walls. The mouse is placed in the device with its back toward the objects from an equidistant position. A camera and software are used to record the exploration time of the mouse for each object (contact with the object by mouth or nose and approaching the object within approximately 2-3 cm range are considered exploration of the object). Within 5 min (many experiments have proven that after 2 min of familiarization period, animals already show good preference for novel objects, and of course, 3 min of familiarization makes the preference more obvious), the number of times, time, and distance the animal explores each object are measured. The third stage is testing period. 1 h after the completion of the second stage is generally selected as the time interval for memory detection (longer time intervals may be used to evaluate the effect of memory improvement). One of the two identical objects is replaced with a different object in the device (AC or BC), and similarly, the mouse is placed in the device with its back toward the objects from an equidistant position for 5 min.
b) Water maze experiment: The Morris water maze includes a circular pool diameter of 120 cm, height 60 cm; a columnar plexiglass platform diameter of 10 cm, height 40 cm. The water level in the pool is about 45 cm high, and both room and water temperature are maintained at 22 ±2° C. The platform is placed at the center position of a certain quadrant, submerged about 2 cm below the water surface. During training, the mouse is gently placed in the water from a ½ radian position of any quadrant with its head facing the pool wall, and its latency period (the time taken by the mouse to find the platform from entering the water) and path are detected as indicators to measure the mouse's learning memory and test performance. Each mouse is trained 3 times in 3 quadrants daily, with each swimming time limited to 90 s. If the platform is not found within 90 s, the system automatically stops recording, and the latency period is recorded as 90 s. The tester then guides it onto the platform, and after a 20 s rest, the next training begins. The initial training period is 6 days, with daily recording of the time needed for mice to find the platform, recorded as the latency period required for mice to find the platform. After one day of rest, the platform is removed, and the time spent by mice in the target quadrant within 60 s is calculated, and the number of crossings at the platform position is analyzed to evaluate their memory ability.
2) Other behavioral detection of mice: open field, conditional fear experiment;

Open field experiment: The open field experiment is an experiment in which animals are placed in a closed open metal box area with a length of 80 cm, width of 80 cm, and height of 50 cm to observe mouse activity. This area is divided into squares, and typically the number of squares crossed by mice within a given time is counted to examine the anxiety emotional state and movement ability of the experimental mice. First, before the experiment, the mice need to be placed in the open field experiment room for 1-2 days, with clear day and night distinction. The experimental area is set up on the computer, the experimental area is divided equally into a “ [symbol] ” shape, with the innermost circle as the central region and the outermost as the peripheral region, then the camera is turned on, the light source is adjusted, and information such as the position, movement route, and distance of the experimental mice is recorded using the computer. Recording is stopped after observing for a certain time, generally about 5 minutes.
3) Quantify mouse hippocampal related protein content: immunoblotting;
4) Disrupting the binding of Syn2b and GluA2 in HEK 293T cells: co-immunoprecipitation, immunoblotting.

### 3. Experimental Results

*P<0.05, compared with control group

**P<0.01, compared with control group

***P<0.001, compared with control group

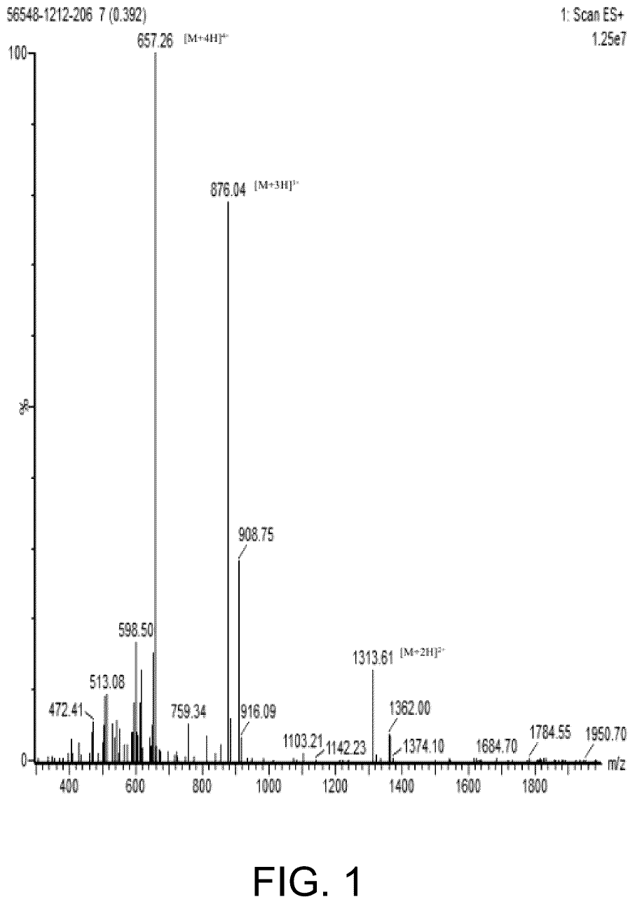

The sequence of TAT-P6 is shown in SEQ ID NO:3, artificially synthesized by Jiangsu Qiangyao Biotechnology Co., Ltd., and the mass spectrometry analysis is shown in FIG. 1. The analysis results show that the amino acid sequence of the synthesized peptide is consistent with SEQ ID NO:3. The purity of the synthesized TAT-P6 peptide is 95.03%, 10 mg per vial, in white powder form, which may be completely dissolved in water, and sealed and stored away from light at-20° C. The sequence of TAT-S6 is shown in SEQ ID NO:4, also synthesized by the same company.

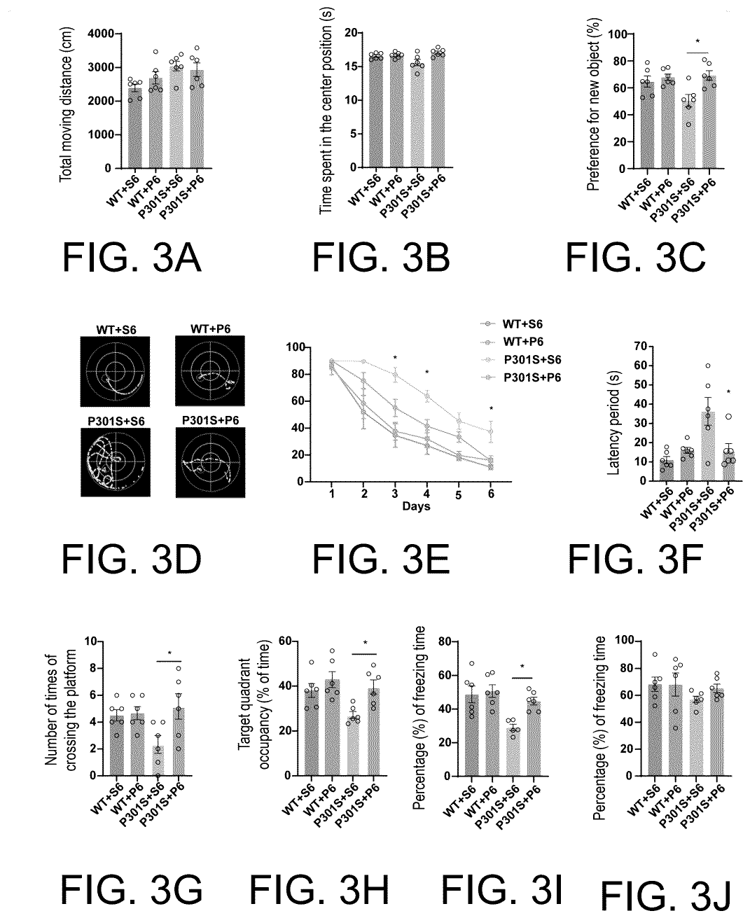

Peptide changes learning and memory impairment in mice: see FIG. 3A to FIG. 3J. C57 and P301S mice were given 15 mg/kg TAT-P6 peptide solution, while control mice were subcutaneously injected with 15 mg/kg TAT-S6 peptide solution, continuously injected for 14 days. Behavioral detection was conducted again, and we discovered that P301S mice injected with 15 mg/kg TAT-P6 solution showed significantly improved learning and memory compared with P301S mice given TAT-S6 peptide solution.

### Example 2

The present disclosure, through selecting C57 mice and P301S mice for subcutaneous injection of TAT-P6 solution, may effectively block the biological action of Syn2b binding with GluA2, enabling the increase of AMPA receptor membrane expression, elevated neuronal excitability and enhanced synaptic transmission.

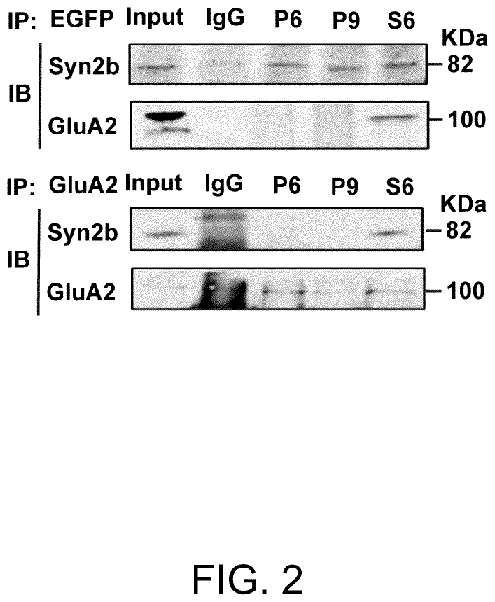

Disrupting the binding of Syn2b and GluA2 in cell experiments: see FIG. 2, plasmids expressing Syn2b and AMPA receptor subunit (GluA2) were co-transfected in HEK 293T cells. After 48 hours of transfection, cells were treated with 10 μM TAT-P6 or 10 μM TAT-S6 for 90 minutes, and then cell proteins were collected. The expressed Syn2b protein was a fusion protein of Syn2b and GFP. Anti-GFP antibody was used to precipitate cell proteins, and anti-GluA2 antibody was used to detect the precipitated proteins. The interaction between GluA2 and Syn2b was shown by immunoblotting on the NC membrane. The results show: after giving 10 μM TAT-P6, the anti-GluA2 antibody could hardly detect the corresponding protein on the NC membrane, suggesting that TAT-P6 blocked the interaction between Syn2b and GluA2. In the control group given TAT-S6, GluA2 protein could be clearly detected, indicating that TAT-S6 could not block the interaction between Syn2b and GluA2.

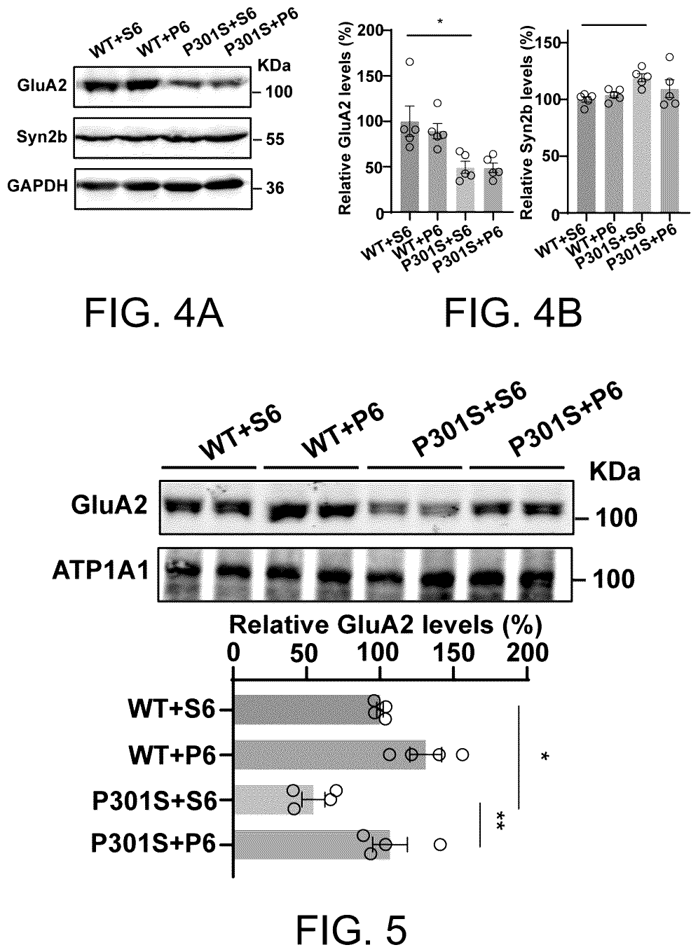

Quantification of Syn2b and GluA2 in mouse hippocampal brain region: see FIG. 4A and FIG. 4B. After behavioral detection was completed in mice, tissue proteins were extracted and immunoblotting was conducted, which discovered that subcutaneous injection of 15 mg/kg TAT-P-6 did not affect the protein content of Syn2b and GluA2.

Quantification of GluA2 on cell membrane in mouse hippocampal brain region: see FIG. 5. Membrane proteins and cytoplasmic proteins were separated from mouse hippocampal tissue. Through immunoblotting, it was discovered that the GluA2 protein on the cell membrane of mice in the TAT-P6 peptide solution treatment group was significantly higher than that in the control group.

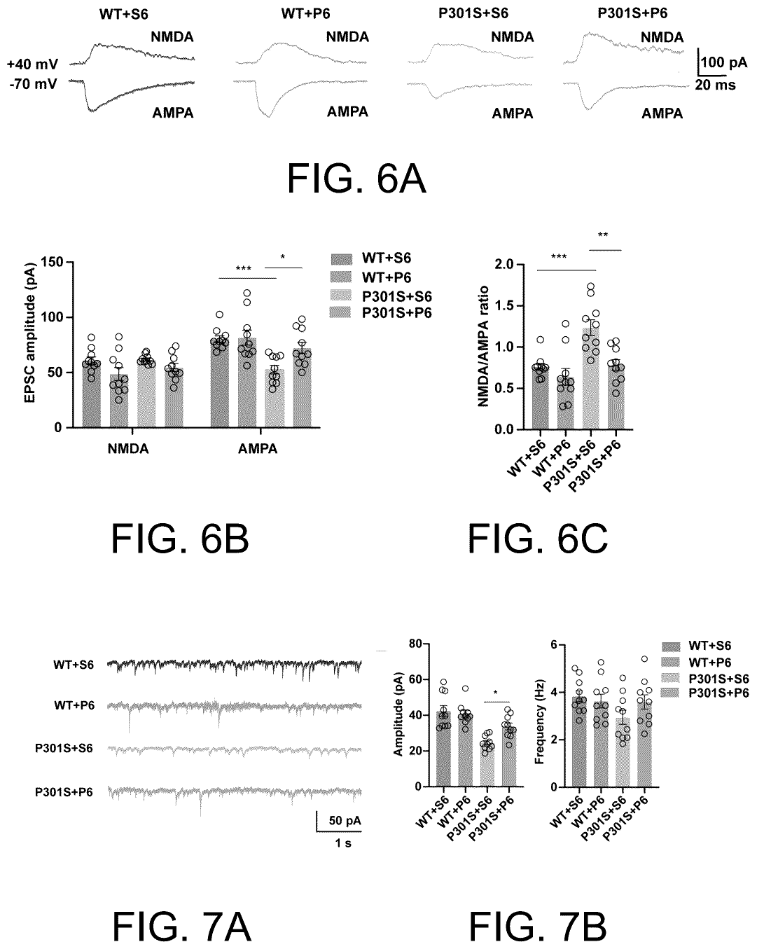

Electrophysiological recording results: see FIG. 6A to FIG. 6C, FIG. 7A and FIG. 7B. During brain slice incubation, 10 μM TAT-P6 solution or 10 μM TAT-S6 solution was given for 30 min treatment, followed by recording of AMPA current and NMDA current in the hippocampal brain region. It was discovered that the AMPA current amplitude increased in the 10 μM TAT-P6 solution group, while NMDA current showed no significant change, and the NMDA/AMPA ratio significantly decreased. When recording spontaneous excitatory postsynaptic potentials (sEPSC), it was found that the amplitude of sEPSC significantly increased after treatment with 10 μM TAT-P6 solution.

### Conclusion

The above results show that in P301S mice, through injection of TAT-P6 solution, the latency period in the Morris water maze was significantly shortened, the time and distance spent in the target quadrant and the number of platform crossings significantly increased, and learning memory ability was enhanced, indicating that it may significantly improve learning memory behavior. Immunoblotting and immunoprecipitation detection results suggest that TAT-P6 may disrupt the binding of Syn2b with GluA2 both in vivo and in vitro, increase GluA2 membrane expression, and electrophysiological experiments prove elevated neuronal excitability and enhanced synaptic transmission. TAT-P6 peptide may be used to improve learning memory ability in Alzheimer's disease.

Those skilled in the art will easily understand that the above description is merely a preferred embodiment of the present disclosure and is not intended to limit the present disclosure. Any modifications, equivalent substitutions, and improvements made within the spirit and principles of the present disclosure should be included within the scope to be protected by the present disclosure.

What is claimed is:

1. An application of a peptide in preparing drugs for treating and/or preventing cognitive impairment, wherein an amino acid sequence of the peptide is shown in SEQ ID NO:1.

2. The application according to claim 1, wherein the cognitive impairment is perception impairment, memory impairment, or thinking impairment.

3. The application according to claim 2, wherein the perception impairment is sensory hypersensitivity, sensory dullness, internal sensory discomfort, sensory degeneration, sensory deprivation, pathological illusion or hallucination, or comprehensive perception impairment;
the memory impairment is memory hypermnesia, memory deficit, or memory error;
the thinking impairment is abstract generalization process impairment, association process impairment, logical thinking impairment, or delusion.

4. The application according to claim 1, wherein the cognitive impairment is cognitive impairment caused by Alzheimer's disease, Lewy body dementia, frontotemporal dementia, Parkinson's disease, or Huntington's chorea;
or cognitive impairment after traumatic brain injury, cognitive impairment after cerebrovascular disease cognitive damage, or postoperative cognitive impairment;
or cognitive impairment caused by progressive supranuclear palsy, corticobasal degeneration, argyrophilic grain disease, Pick's disease, neurasthenia, hysteria, skepticism, menopausal syndrome, depression, obsessive-compulsive disorder, schizophrenia, reactive psychosis, paranoid psychosis, mania, or manic depression.

5. The application according to claim 1, wherein the peptide is used to block binding of Syn2b protein with GluA2 protein, thereby enabling an increase of GluA2 protein membrane expression.

6. The application according to claim 1, wherein the peptide is used to promote an increase of neuronal AMPA current, thereby enabling an increase of spontaneous excitatory postsynaptic current.

7. The application according to claim 1, wherein an N-terminus of the peptide is further connected with a membrane peptide.

8. The application according to claim 7, wherein an amino acid sequence of the membrane peptide is as shown in SEQ ID NO:2.

9. A peptide prepared for treating and/or preventing cognitive impairment, wherein an amino acid sequence of the peptide is as shown in SEQ ID NO:1.

10. The peptide prepared for treating and/or preventing cognitive impairment according to claim 9, wherein an N-terminus of the peptide is further connected with a membrane peptide; an amino acid sequence of the membrane peptide is as shown in SEQ ID NO:2.
